(19) United States
(12) Patent Application Publication
(10) Pub. No.: US 2011/0178476 A1
(43) Pub. Date: Jul. 21, 2011

(54) DRIP DETECTOR WITH MULTIPLE SYMMETRIC SENSORS AND SIGNAL TRANSMISSION BY ZIGBEE NETWORK

(75) Inventor: Ming-Jeng Lin, Taipei (TW)

(73) Assignee: GWG INTERNATIONAL INC., Taipei (TW)

(21) Appl. No.: 12/689,230

(22) Filed: Jan. 19, 2010

(51) Int. Cl. A61M 5/168; H04B 7/005

(52) U.S. Cl. 604/253; 455/41.3; 604/31

(57) ABSTRACT

A drip detector for a tube with multiple symmetric sensors and signal transmission utilizes a ZigBee network and includes a monitoring display host, a drip detector, a stopper and a base. The detector generates signals and alarms through the ZigBee network when it detects changes in drip speed or drip quantity.

### BACKGROUND OF THE INVENTION

1. Field of the Invention

The present invention relates to a drip detector for a tube with multiple symmetric sensors and signal transmissions utilizing a ZigBee network, which generates signals and alarms through the ZigBee network when it detects changes in drip speed or drip quantity.

2. Description of the Related Art

Developments in medical technology and equipment provide increasing protection for patients. IV catheters are a very common tool in hospital environments, and a drip adjuster is used for controlling the IV dripping speed. Typically, the nursing staff adjusts the drip adjuster based only on naked eye observations and experience and also estimates the completion time for each IV bag based on experience. However, the nursing staff may miss the time to change the IV bag, which can lead to medical complaints.

Therefore, it is desirable to provide a drip detector for a tube with multiple symmetric sensors and signal transmissions utilizing a ZigBee network, which generates signals and alarms through the ZigBee network when it detects changes in drip speed or drip quantities to mitigate and/or obviate the aforementioned problems.

### SUMMARY OF THE INVENTION

An objective of the present invention is to provide a drip detector for a tube with multiple symmetric sensors and signal transmission utilizing a ZigBee network.

Another objective of the present invention is to provide a drip detector for a tube with multiple symmetric sensors and signal transmission utilizing a ZigBee network, which has a drip detector with a plurality of IR sensing modules.

Another objective of the present invention is to provide a drip detector for a tube with multiple symmetric sensors and signal transmission utilizing a ZigBee network, which has the IR sensing modules composed of the IR LED and the IR sensor.

Another objective of the present invention is to provide a drip detector for a tube with multiple symmetric sensors and signal transmission utilizing a ZigBee network, which has USB connections between the drip detector, the stopper and the monitoring display host.

Other objects, advantages, and novel features of the invention will become more apparent from the following detailed description when taken in conjunction with the accompanying drawings.

### BRIEF DESCRIPTION OF THE DRAWINGS

FIG. 1 is a perspective view of a drip detector for a tube with multiple symmetric sensors and signal transmissions utilizing a ZigBee network according to an embodiment of the present invention.

FIG. 2 is an exploded perspective view of a drip detector for a tube with multiple symmetric sensors and signal transmissions utilizing a ZigBee network according to an embodiment of the present invention.

FIG. 3A is rear view of a drip detector for a tube with multiple symmetric sensors and signal transmissions utilizing a ZigBee network according to an embodiment of the present invention.

FIG. 3B is a top view of a drip detector for a tube with multiple symmetric sensors and signal transmissions utilizing a ZigBee network according to an embodiment of the present invention.

FIG. 4A is an Z axis sensing schematic drawing of a drip detector for a tube with multiple symmetric sensors and signal transmissions utilizing a ZigBee network.

FIG. 4B is an X and Y axis sensing schematic drawing the drip detector for a tube with multiple symmetric sensors and signal transmissions utilizing a ZigBee network.

FIG. 5A is a schematic drawing of a stopper of a drip detector for a tube with multiple symmetric sensors and signal transmissions utilizing a ZigBee network according to an embodiment of the present invention.

FIG. 5B is a cross-sectional view of a stopper of a drip detector for a tube with multiple symmetric sensors and signal transmissions utilizing a ZigBee network according to the present invention.

FIG. 6 is a block drawing of a drip detector for a tube with multiple symmetric sensors and signal transmissions utilizing a ZigBee network according to an embodiment of the present invention.

### DETAILED DESCRIPTION OF THE PREFERRED EMBODIMENT

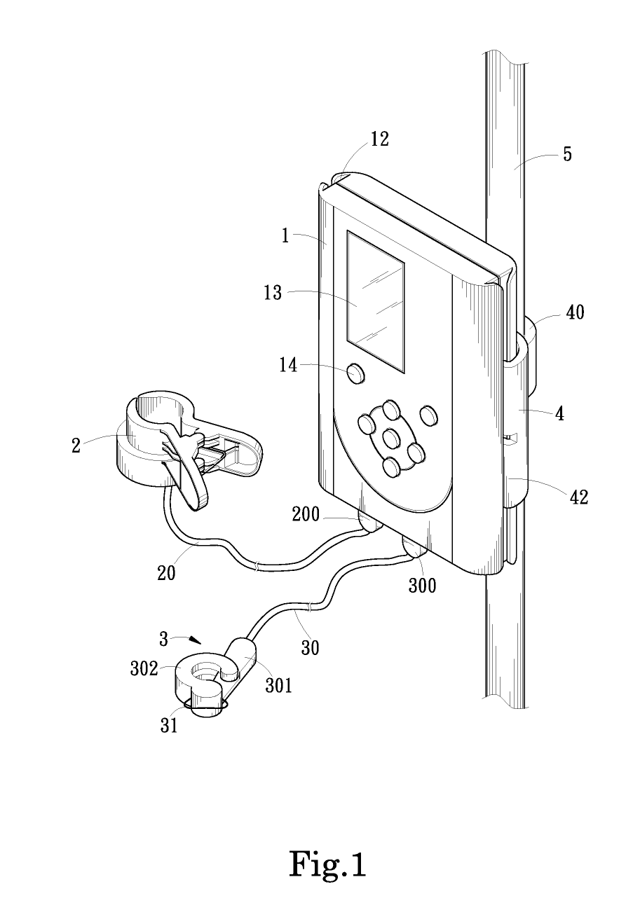

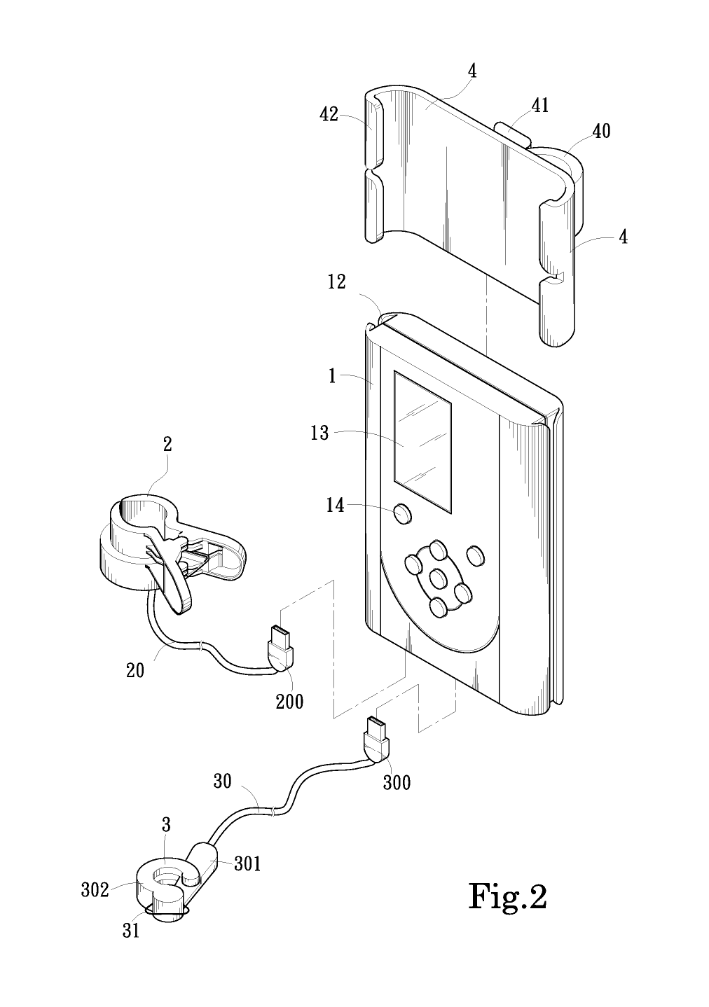

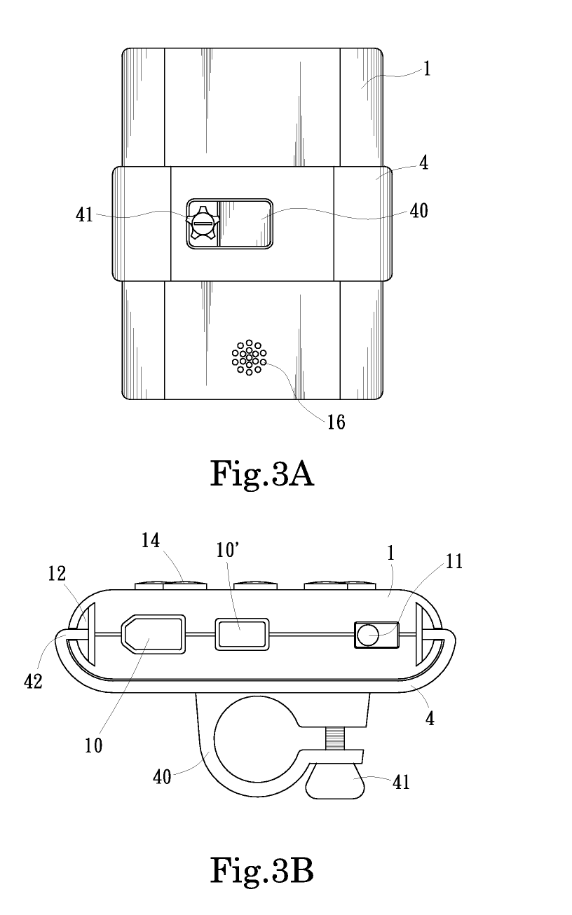

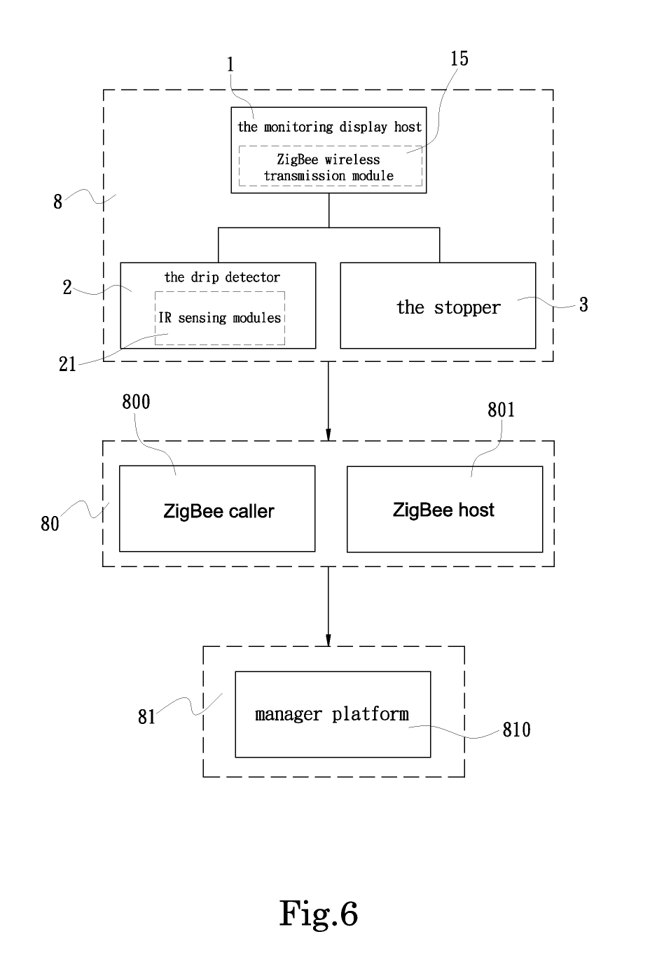

First please refer to FIGS. 1 and 2. A drip detector for a tube with multiple symmetric sensors and signal transmissions utilizing a ZigBee network comprises a monitoring display host 1, a drip detector 2, a stopper 3 and a base 4. The monitoring display host 1 is substantially rectangular and has a sliding slot 12 on two opposite sides engaging with a fastening member 42 of the base 4 to secure the monitoring display host 1. Additionally, one end of the monitoring display host 1 has connecting ports 10, 10′ and a socket 11. The connecting ports 10, 10′ are USB interface connecting ports and are used for connecting to wires 20, 30 of the drip detector 2 and the stopper 3, and the socket 11 (not shown in this drawing; refer instead to FIG. 3B) is used for supplying power. A display 13 and a plurality of buttons 14 are disposed on one side of the monitoring display host 1 for monitoring received data and settings. Moreover, the monitoring display host 1 has a ZigBee wireless transmission module 15 for transmitting the received signals and data to a ZigBee caller 800, a ZigBee host 801 and a managing platform 810 (not shown in this drawing; refer instead to FIG. 6).

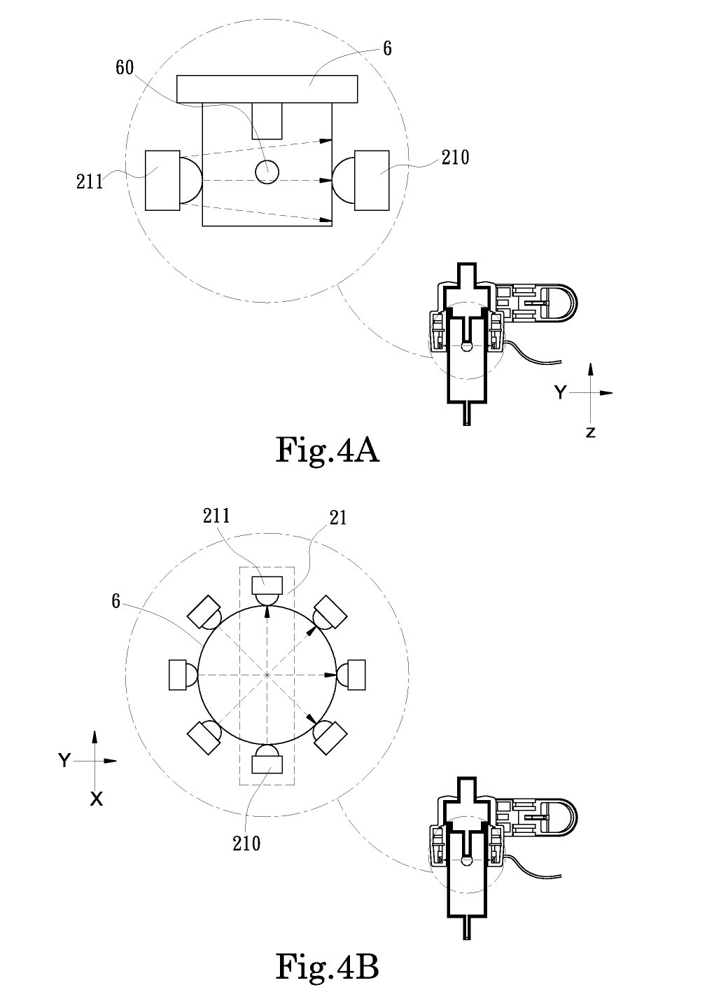

The drip detector 2 is similar to a clip and includes the connecting wire 20 with a USB plug 200 for connection to the connecting port 10 of the monitoring display host 1. The drip detector 2 comprises a plurality of IR sensing modules 21, and each IR sensing module 21 is includes an IR LED 211 and an IR sensor 210 (not shown in this drawing; refer instead to FIGS. 4A, 4B). With the correspondence between the signal transmissions and the receiving device and the plurality of sets of modules in the drip detector 2, the time differences and dripping speeds among drip events can be accurately calculated. The detected data is transmitted to the monitoring display host 1 through the connecting wire 20, and the ZigBee wireless transmission module 15 transmits the signal to the corresponding receiving devices.

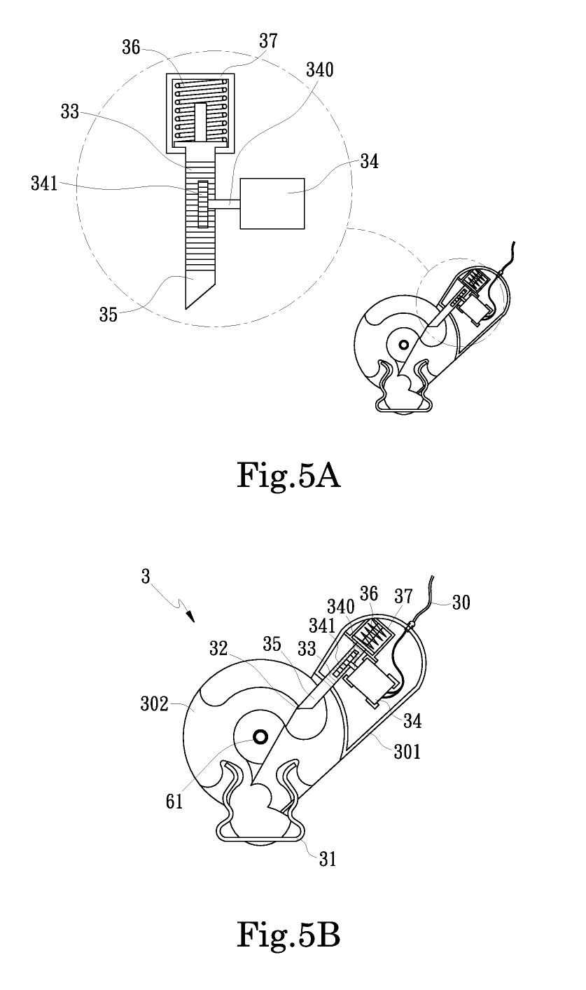

The stopper 3 is a fastening device and includes a hook member 302, a pushing member 301 and a spring 31. One end of the pushing member 301 has a connecting wire 30 with a USB plug 300 for connection to the connecting port 10 of the monitoring display host 1. The spring 31 provides a connection between the hook member 302 and the pushing member 301 and an elastic force to rotate the hook member 302. Additionally, the pushing member 301 has a securing latch 35, a spring 36, and a motor 34. The securing latch 35 is knife-shaped and has a serrated shaft 33 on its surface for engaging with the gear 341 of the motor 34, and one end of the latch 35 is attached to a holder 37. The holder 37 further includes a spring 36 enabling stretching of the securing latch 35. One end of a rotation shaft 340 of the motor 34 has a gear 341 engaged with the serrated shaft 33 of the securing latch 35, and with the rotation of the gear, the securing latch 35 is driven to be stretched (not shown in this drawing; refer instead to FIGS. 5A, 5B).

The base 4 has a C-like shape and a hook 42 at each of its ends corresponding to the sliding slots 12 of the monitoring display host 1. The base 4 further comprises a securing jacket 40 and a button 41 (as shown in FIG. 3B) for securing to a support frame 5 and holding the monitoring display host 1 at different heights.

With the detection provided by the IR sensing module 21 (not shown in this drawing; refer instead to FIGS. 4A, 4B), the plurality of IR LEDs 211 and the IR sensors 210 are cross matched with each other to accurately detect the time difference among drip events to obtain data in X, Y and Z directions. The ZigBee wireless transmission module of the monitoring display host 1 has characteristics such as low power consumption, low cost, high node capacity, flexible working frequency, high safety and mobility, and the transmission manner of its network can be star shaped, tree shaped, or web shaped. The entire network equipments includes a primary host and a plurality of sub-hosts, which join the network through the host.

Please refer to FIGS. 3A and 3B. As shown in the drawings, the base 4 utilizes the two hooks 42 for engaging with the sliding slots 12 of the monitoring display host 1. The monitoring display host 1 further includes a bee buzzer 16 for signaling an alarm to others using sound or light. One end of the monitoring display host 1 has connecting ports 10, 10′ and a socket 11. The connecting ports 10, 10′ are USB interface connecting ports and are used for connecting to wires 20, 30 of the drip detector 2 and the stopper 3 and transmitting the detected data to the monitoring display host 1, which is sent through the ZigBee wireless transmission module. Meanwhile, the stopper 3 is activated to pinch the tube and stop the dripping movement.

Please refer to FIGS. 4A and 4B. The IR sensing module 21 of the drip detector 2 executes drip monitoring. The drip detector 2 is mounted onto a drip chamber 6 such that the IR sensing module 21 surrounds the drip chamber 6 (as shown in FIGS. 4A and 4B). The vertical axis is defined as the Z direction and the horizontal axis is defined the X direction. When a drip 60 falls and passes through the IR sensing module 21, the plurality of IR LEDs 211 and the IR sensors 210 of the IR sensing module 21 provide an IR light web to detect each falling drip 60. When the IR light is interrupted, the sensor outputs no signal; and if the IR light is not interrupted over a predetermined time period, the sensor outputs an error signal. However, during operations, if the detector is shaken, the plurality of IR sensing modules 21 of the drip detector 2 provides an IR web for the X and Y axial directions, to reduce drip miscalculations during shaking.

Please refer to FIGS. 5A and 5B. The stopper 3 has the pushing member 301 and the hook member 302 and the spring 31 combines them together and provides an elastic force. However, the pushing member 301 includes the securing latch 35, the spring 36, and the motor 34. The securing latch 35 has a knife shape and the serrated shaft 33 on its surface for engaging with the gear 341 of the motor 34, and one end of the latch 35 is attached to a holder 37. The holder 37 further includes a spring 36 enabling the securing latch 35 to be stretched. One end of a rotation shaft 340 of the motor 34 has a gear 341 engaged with the serrated shaft 33 of the securing latch 35. When the detected signal from the drip detector 2 is transmitted to the monitoring display host 1, the ZigBee wireless transmission module 15 (not shown in this drawing, please refer instead to FIG. 6) simultaneously sends the signals to the stopper 3. After the stopper 3 receives the signals, the motor 34 is activated to move the securing latch 35 to release the securing latch head 32; with the elastic force provided by the spring 31, the securing latch head 32 rotates to close the tube 61 and prevent air from entering further.

Please refer to FIG. 6. An embodiment drip detector disclosed in the present invention has three end units; one is a front end unit 8, one is a communication unit 80, and the other one is a back end unit 81. The front end unit 8 is used for signal detection and display purposes, which includes the monitoring display host 1, the drip detector 2 and the stopper 3. The monitoring display host 1 has a ZigBee wireless transmission module 15, and the drip detector 2 has a plurality of IR sensing modules 21. When the IR sensing module 21 of the drip detector 2 detects a change among the drip speed or drip quantity, an indicating signal is sent to the monitoring display host 1 and the stopper 3 to close the tube 61, and is transmitted to the communications unit 80 and the back end unit 81 via the ZigBee wireless transmission module 15. The communications unit 80 is used for signal transmission purposes. When the signal is generated, the ZigBee wireless transmission module 15 transmits the signal to the ZigBee caller 800 or the ZigBee host 801. Usually, the ZigBee caller 800 can be carried around by a member of the nursing staff or a family member, and the signal is recorded in the ZigBee host 801. The back end unit 81 is used for managing and recording functions. If the generated signal is not properly cleared, the manager platform 810 can provide notification of the generated signals and manage a large area through the ZigBee wireless transmission network.

Although the present invention has been explained in relation to its preferred embodiment, it is to be understood that many other possible modifications and variations can be made without departing from the spirit and scope of the invention as hereinafter claimed.

What is claimed is:

1. A drip detector for a tube with multiple symmetric sensors and signal transmissions utilizing a ZigBee network comprising: a monitoring display, a drip detector, a stopper and a base, characterized in that:
the monitoring display host comprises a ZigBee wireless transmission module, the drip detector comprises an IR sensing module;
wherein with the drip detector detects time differences and dripping speeds among drip events, and when the time differences and dripping speeds among drip events change, the ZigBee wireless transmission module transmits signals to corresponding callers and hosts, and the stopper stops a liquid supply from the tube.

2. The drip detector for a tube with multiple symmetric sensors and signal transmission utilizing a ZigBee network as claimed in claim 1, wherein the monitoring display host further comprises a vibration module, a bee buzz module and a lighting module.

3. The drip detector for a tube with multiple symmetric sensors and signal transmission utilizing a ZigBee network as claimed in claim 1, wherein the IR sensing module is an IR LED or an IR sensor.

4. The drip detector for a tube with multiple symmetric sensors and signal transmission utilizing a ZigBee network as claimed in claim 1, wherein the drip detector has at least one IR sensing module.

5. The drip detector for a tube with multiple symmetric sensors and signal transmission utilizing a ZigBee network as claimed in claim 4, wherein the IR sensing module is capable of detecting a falling liquid drop in X, Y, Z directions.

6. The drip detector for a tube with multiple symmetric sensors and signal transmission utilizing a ZigBee network as claimed in claim 1, wherein the monitoring display host further comprises an RS485 cable transmission module.

7. The drip detector for a tube with multiple symmetric sensors and signal transmission utilizing a ZigBee network as claimed in claim 1, wherein the stopper comprises a pushing member and a hook member.

8. The drip detector for a tube with multiple symmetric sensors and signal transmission utilizing a ZigBee network as claimed in claim 6, wherein the pushing member and the hook member are secured with a first spring.

9. The drip detector for a tube with multiple symmetric sensors and signal transmission utilizing a ZigBee network as claimed in claim 6, wherein the pushing member further comprises a second spring, a securing latch, a motor and a gear.

10. The drip detector for a tube with multiple symmetric sensors and signal transmission utilizing a ZigBee network as claimed in claim 6, wherein the hook member forms a securing latch head.
